(19) United States
(12) Patent Application Publication
(10) Pub. No.: US 2009/0048328 A1
(43) Pub. Date: Feb. 19, 2009

(54) Compound Comprising Prodigiosin From Serratia Macescence B-1231 Kctc 0386Bp for Prevention and Treatment of Acute Graft-Versus-Host Disease

(75) Inventors: Hwan Mook Kim, Daejeon (KR); Sang-Bae Han, Chungcheongbuk-do (KR); Chang-Woo Lee, Daejeon (KR); Ki-Hoon Lee, Daejeon (KR); Sung-Kyu Park, Daejeon (KR); Ki-Ho Lee, Seoul (KR); Jong-Soon Kang, Daejeon (KR); Won-Kee Yoon, Daejeon (KR); Young-Kook Kim, Daejeon (KR)

Correspondence Address: BUCHANAN, INGERSOLL & ROONEY PC, POST OFFICE BOX 1404, ALEXANDRIA, VA, 22313-1404, US

(73) Assignee: Korea Research Institute of Bioscience and Biotechnology, Daejeon (JP)

(21) Appl. No.: 11/988,561

(22) PCT Filed: Jul. 10, 2006
(86) PCT No.: PCT/KR2006/002700

§ 371 Date: Jan. 10, 2008

(30) Foreign Application Priority Data
Jul. 11, 2005 (KR) 10-2005-0062287

(51) Int. Cl. A61K 31/4155; A61P 37/06

(52) U.S. Cl. 514/422

(57) ABSTRACT

This invention relates to a composition for the prevention and treatment of acute graft-versus-host disease comprising prodigiosin isolated from Serratia marcescence B-1231 KCTC 0386BP, as an effective ingredient. The prodigiosin is immunosup-pressive by selectively suppressing the proliferation of T-cells through the suppression of the expression of IL-2 receptors, which is needed for the activation of T-cells. Prodigiosin can be used either alone or in conjunction with cyclosporin A for greater effect, since the two substances have different mode of actions. These make prodigiosin effective for the prevention and treatment of acute graft-versus-host disease.

### TECHNICAL FIELD

This invention relates to a composition for the prevention and treatment of acute graft-versus-host disease comprising prodigiosin isolated from Serratia marcescence B-1231 KCTC 0386BP, as an effective ingredient.

### BACKGROUND ART

Acute graft-versus-host disease is a complex disease that appears when T-cells with immune function among bone marrow cells attack the transplanted organ of the patient. Major symptoms are known to appear in the skin, liver and the digestive system.

The disease emerges in three phases. The first phase emerges prior to the bone marrow transplant; the patient's tissues are harmed, and in some cases, antigen-presenting cells are activated due to bacterial infection. In the second phase, the T-cells among the transplanted bone marrow cells are activated. The patient's antigen-presenting cells, which have already been activated, specialize the T-cells into Th1 cells, and ultimately produce cytokines such as IL-2 and IFN-gamma. In the final phase, the patient's organ is disrupted. When the cytotoxic T-cells and natural killer cells are activated by the cytokine that is secreted from the activated Th1 cells, they attack the organ of the patient and generate acute graft-versus-host disease.

Several methods have been suggested to treat acute graft-versus-host disease. Some of the suggested methods are as follows: removing T-cells from the bone marrow cells that are being transplanted, administering antibodies to CD80 and CD86 in order to control the responses of the T-cells and antigen-presenting cells, administering antibodies against cytokines such as IL-2 and IFN-gamma, or administering compound immunosuppressive such as cyclosporin A, rapamycin and FK-506 steroid medicine. Among these methods, administering compound immunosuppressive to restrain the activation of T-cells has been the most widely adopted.

A wide range of compound immunosuppressive has been developed at present. Among these, cyclosporin A has shown the most excellent clinical effects, and has been widely used to treat autoimmune diseases, organ transplant rejection and various inflammatory diseases. When a large volume of cyclosporin A is used, it can perfectly suppress the activation of T-cells and treat the disease. However, it will also cause serious side effects, including kidney toxicity. Thus, it is recommended that only a small amount of cyclosporin A be administered. In order to supplement the reduced medical effect due to the reduced dose, a few different immunosuppressive are administered in conjunction with cycloporin A. A prerequisite for the combined use of cyclosporin A and other immunosuppressive is that their mode of action and toxicity regions should be different. R&D efforts are still being made to develop immunosuppressive for use in combination with cyclosporin A that satisfy this condition.

Microbes in Streptomyces and Serratia produce red substances with pyrolline pyromethene structures. Among these are prodigiosin, metacycloprodigiosin, prodigiosen, desmethoxiprodigiosin and prodigiosin 25-C. These materials are known to have antibiotic, anti-malarial and anti-cancer effects.

The disease-treating effects of prodigiosin have already been reported by the inventors of this invention (Korea Patent No. 150106848 and 190392225, International Journal of Immunopharmacology 20, pp 1-13, 1997; Journal of Pharmacology and Experimental Therapeutics 299(2), pp 415-425, 2001).

However, no mention has been made in the aforementioned documents regarding the use of prodigiosin for the treatment of acute graft-versus-host disease.

Thus, the inventors of this invention have completed this invention by confirming the excellent immune-suppression effect for acute graft-versus-host disease using prodigiosin isolated from Serratia marcescene B-1231 KCTC 0386BP, alone or in combination with cyclosporin A.

### DISCLOSURE

### Technical Problem

The object of this invention is to provide a composition for the prevention and treatment of acute graft-versus-host disease comprising prodigiosin isolated from Serratia marcescence B-1231 KCTC 0386BP, as an effective ingredient.

### Technical Solution

To achieve the abovementioned object, this invention provides a composition for the prevention and treatment of acute graft-versus-host disease comprising prodigiosin isolated from Serratia marcescence B-1231 KCTC 0386BP, as an effective ingredient.

Since the mode of action of prodigiosin is different from that of cyclosporin A, the most commonly used immunosuppressive, the two can be used in combination for the prevention and treatment of acute graft-versus-host disease.

Below is the detailed disclosure of this invention;

Prodigiosin used in this invention can be isolated from Serratia marcescence B-1231 KCTC 0386BP according to the method stated in Korea Patent No. 252197, which is as follows:

To produce the immunosuppressant, a 1 l Erlenmeyer flask was prepared with culture medium (1% soluble starch, 0.5% phamamedia, 0.2% glucose, 0.1% ammonium sulfate, 0.1% potassium phosphate, 0.05% MgSO₄.7H₂O, 0.1% calcium chloride, 0.3% sodium chloride, with the initial pH at 7.0), and 20˜100 ml of Serratia marcescence B-1231 KCTC 0386BP was added and cultivated for 50 to 70 hours at a temperature of 20-30° C.

To extract the activated substances, the same amount of ethyl acetate as that of the fermented solution was added and stirred for 20 to 60 minutes. The organic solvent layer was collected and concentrated at lower pressure, from which a red substance was attained. Then, the activated substance was attained through the solvent gradient method by processing the chloroform:methanol solution in the silica gel column. Then, prodigiosin could be isolated by using silica gel thin layer chromatography.

The chemical formula of prodigiosin isolated through the abovementioned method is presented below:

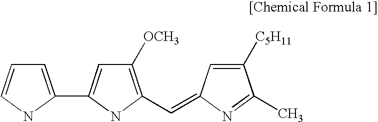

Thus, this invention provides a composition for the prevention and treatment of acute graft-versus-host disease comprising prodigiosin isolated from Serratia marcescence B-1231 KCTC 0386BP, as an effective ingredient.

In addition, the invention provides the composition for the prevention and treatment of acute graft-versus-host disease characterizing the possibility of administration in combination the said prodigiosin with cyclosporin A due to differences in mode of action.

The composition for the prevention and treatment of acute graft-versus-host disease includes 0.1˜50% of the above described substance in the total weight of the composition.

The medical composition comprising the substance in this invention may also include carriers, excipients or attenuants that are ordinarily used for the manufacture of pharmaceutical compositions.

As for the form of pharmaceutical administration of the compound in this invention, it can be administered in the form of salts, alone, in combination with other pharmaceutically active compounds, or in the form of appropriate collection.

The pharmaceutical composition of this invention may be processed for oral administration in the form of powders, granules, tablets, capsules, suspensions, emulsions, syrups or aerosols. It can also be processed as medicine for application, suppository or sterilized injection solutions. Carriers, excipients or attenuants that can be included in the compound in this invention include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrollidone, water, methylhydrobenzoate, propylhydroxybenzoate, talc, magnesium stearate and mineral oils. For pharmaceutical manufacture, commonly used excipients and attenuants such as fillers, diluents, bonding agents, wetting agents, disintegrants and surfactants can be used. As for solid forms for oral medication, the medicine can be manufactured in the form of powders, granules, tablets, capsules and suspensions, and one or more excipients are usually mixed, such as starch, calcium carbonate, sucrose, lactose or gelatin. Apart from these excipients, lubricants such as magnesium stearate and talc can also be used. Suspension, solution, emulsion and syrup are liquid types of oral medicines, and on top of commonly used attenuants such as water or liquid paraffin, various excipients such as wetting agents, sweeteners, aromatics and preservatives can also be included. Non-oral medicines can be made in the form of sterilized aqueous solutions, non-aqueous solvents, emulsions, lyophilized medicines or suppositories. Non-aqueous solvents and suspensions can be vegetable oils such as propylene glycol, polyethylene glycol and olive oil, as well as ester that can be injected, such as ethyloleate. As for suppositories, witepsol, macrogol, tween 61, cacao butter, laurin butter and glycero gelatin can be used.

The preferable dose of the composition of this invention can be determined appropriately according to the state and weight of the patient, degree of disease, type of medicine and its method of administration and duration. However, in order to get the desirable effect of the medicine, it is advisable to administer 0.0001˜100 mg/kg of the compound per day, most desirably 0.001˜10 mg/kg. The daily dose can be administered either at once, or multiple times a day. However, the said dosage does not in any way limit the scope of this invention.

The composition of this invention can be administered to mammals such as rats, mice, animals and people in various methods. All the administration methods can be used including oral medicines, as well as rectal, vein, muscular, hypodermic, endometrial and intracerebroventricular injections.

The details of this invention will be explained in the following execution and experimental examples. However, the examples presented below are introduced simply as examples and do not limit this invention in any terms.

### ADVANTAGEOUS EFFECTS

This invention relates to a composition for the prevention and treatment of acute graft-versus-host disease comprising prodigiosin isolated from Serratia marcescence B-1231 KCTC 0386BP, as an effective ingredient. Whereas the prodigiosin of this invention does not affect the expression of IL-2, it is immunosuppressive by selectively suppressing the proliferation of T-cells through the suppression of the expression of IL-2 receptors that are needed for the activation of T-cells, while not affecting the signal transduction after the IL-2 receptors are combined with the T-cells. Prodigiosin can be used either alone or in conjunction with cyclosporin A, the generally used immunosuppressive, for greater effect since the two substances have different mode of actions. These make prodigiosin effective for the prevention and treatment of acute graft-versus-host disease.

### BRIEF DESCRIPTION OF THE DRAWINGS

FIGS. 1 and 2 refer to the survival rate (FIG. 1) and the weight (FIG. 2) of the Balb/c mouse with acute graft-versus-host disease, which was administered with prodigiosin to confirm its therapeutic effect on the disease;

FIG. 3 indicates the effect of prodigiosin on the proliferation of T-cells;

FIG. 4 shows the effect of cyclosporin A on the proliferation of T-cells;

FIG. 5 shows the effect of prodigiosin and cyclosporin A on the expression of IL-2 of T-cells;

FIG. 6 shows the effect of prodigiosin and cyclosporin A on the expression of IL-2 receptors (IL-2R alpha) of T-cells;

FIG. 7 indicates the relevance of the immunosuppressive effect of prodigiosin to IL-2;

FIG. 8 indicates the relevance between the immunosuppressive effect of cyclosporin A and IL-2;

FIG. 9 displays the effect of prodigiosin on the proliferation of IL-2 dependent T-cells, and;

FIG. 10 shows the effect of cyclosporin A on the proliferation of IL-2 dependent T-cells

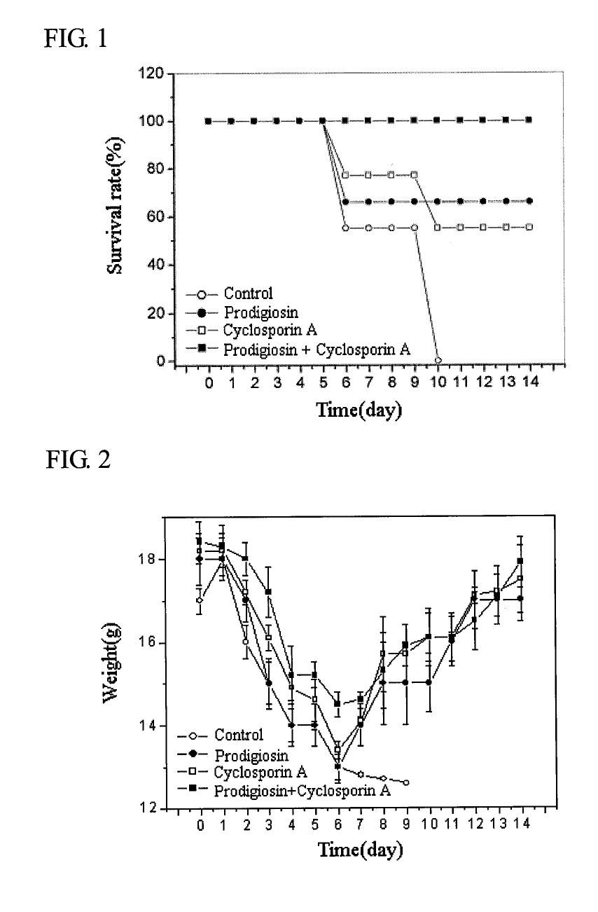

### BEST MODE FOR CARRYING OUT THE INVENTION

### Example 1

### Separation and Purification of Prodigiosin

To produce the immunosuppressive, a 1 l Erlenmeyer flask was prepared with culture medium (1% soluble starch, 0.5% phamamedia, 0.2% glucose, 0.1% ammonium sulfate, 0.1% potassium phosphate, 0.05% MgSO₄.7H₂O, 0.1% calcium chloride, 0.3% sodium chloride, with an initial pH of 7.0), and 100 ml of Serratia marcescence B-1231 KCTC 0386BP was added and cultivated for 62 hours at a temperature of 28° C.

To extract the activated substances, the same amount of ethyl acetate as that of the fermented solution was added and stirred for 30 minutes. The organic solvent layer was collected and concentrated at a lower pressure, from which a red substance was attained. Then, the activated substance was attained through the solvent gradient method by processing the chloroform:methanol solution in the silica gel column. Then, prodigiosin could be isolated by using silica gel thin layer chromatography.

### MODE OF THE INVENTION

### Experimental Example 1

### Therapeutic Effect of Prodigiosin on Acute Graft-Versus-Host Disease

1-1. Experiment on Mouse Survival

The therapeutic effect of prodigiosin in this invention was observed through an experiment of bone marrow transplantation.

To induce acute graft-versus-host disease, bone marrow cells (5×10⁷ cells) and spleen cells (1×10⁷ cells) of the C57BL/6 mouse were transplanted to the Balb/c mouse, which had been irradiated and had lost immune function. After the transplantation of the cells, the survival rate of the mouse was observed for 14 days (Blood, 97(4), pp 1123-1130, 2001).

A 0.5% Tween 80 was given to the mouse in the control group through peritoneal injections every other day; The prodigiosin or cyclosporin A (Sigma Co.), separated and purified in Example 1, was melted in 0.5% Tween 80 to make a 1 mg/kg concentration and administered through peritoneal injections every other day. For the group with combined applications, prodigiosin and cyclosporin A were injected jointly.

As a result of the execution of the experiment described above, 50% of the Balb/c mice in the control group (∘) died six days after the cell transplantation. By the 10th day, all of the Balb/c mice died of acute graft-versus-host disease. However, in the group with prodigiosin (), 35% of the Balb/c mice died six days after the cell transplantation while the rest (65%) survived through to the end of the experiment. In the group with cyclosporin A (□), 50% survived through to the end of the experiment. Meanwhile, in the group with the combination of prodigiosin and cyclosporin A (▪), all of the mice survived.

1-2. Experiment on Weight Changes of Mice

Changes in the weight of the mice were observed in the same manner as described in Experimental Example 1-1.

As seen in FIG. 2, the mice in the control group (∘) rapidly lost weight; all of the mice constantly lost weight up to the 9th day and died. This is a representative symptom of acute graft-versus-host disease that occurs in the way that the T-cells of the transplanted bone marrow cells attack the organ of the host. On the other hand, in the groups with prodigiosin () and cyclosporin A (□), and the group with the combined use of prodigiosin and cyclosporin A (▪), the Balb/c mice started losing weight from the 2nd day and continued to lose weight up to the 6th day. However, they started regaining weight from the 7th day and then recovered normal weight by the 14th day.

From the results of Experimental Examples 1-1 and 1-2, it is noted that when prodigiosin and cyclosporin A were used separately in 1 mg/kg concentration, they showed a 50˜60% therapeutic effect against acute graft-versus-host disease, and that when they were used jointly, the therapeutic effect increased to 100%.

### Experimental Example 2

### Impact Against the Proliferation of T- and B-Cells

2-1. Effect of prodigiosin against the proliferation of T- and B-cells

T-cells were separated from the spleen of the C57BL/6 mouse for the experiment. 1 μg/ml concanavalin A (ConA) was used to generate the proliferation of T-cells, and 1 μg/ml lypopolysaccaride induced proliferation of the B-cells. The prodigiosin separated in Execution Example 1 was melted in dimethyl sulfoxide (DMSO) and was added to the culture medium in a manner to achieve a final concentration of 3, 10, 30 ng/ml. After 60 hours of cultivation, [³H]-thymidine was added to the culture medium with a concentration of 1 μCi/well. After 12 hours, the cells were collected and the amount of radioactivity in the DNA was measured.

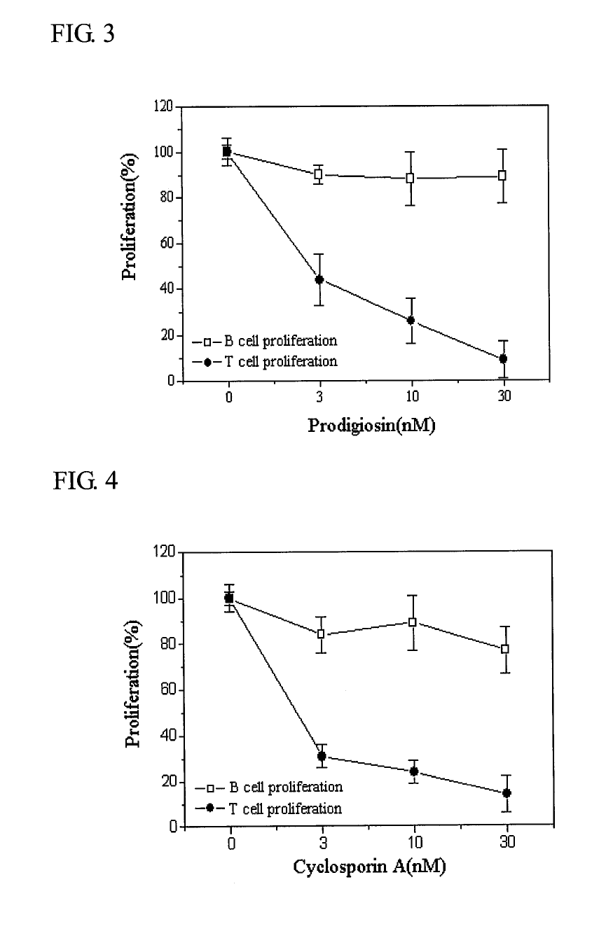

As seen in FIG. 3, the experimental results suggest that prodigiosin selectively suppressed the proliferation of T-cells, whereas it could not suppress the proliferation of B-cells.

2-2. Effect of Cyclosporin a Against the Proliferation of T- and B-Cells

The impact of cyclosporin A (Sigma Co.) on the proliferation of T- and B-cells was observed through the same method described in Experimental Example 2-1.

As seen in FIG. 4, the experimental results indicate that cyclosporin A selectively suppressed the proliferation of T-cells, whereas it failed to suppress the proliferation of B-cells.

Thus, it can be derived from the results of Experimental Examples 2-1 and 2-2 that the prodigiosin and cyclosporin A of this invention are immunosuppressive that selectively suppress the proliferation of T-cells; The two indicated similar remedial effects, since the IC₅₀ (50% suppression concentration) of prodigiosin and cyclosporin A were 2.03 ng/ml and 1.29 ng/ml, respectively.

### Experimental Example 3

### Impact of Prodigiosin and Cyclosporin Against The Proliferation of T- and B-Cells

In order for T-cells to be activated, three types of signaling systems are required; T-cells are activated through interactions between antigen-presenting cells and MHC-TCR (Signal 1) and between antigen-presenting cells and CD80-CD2 (Signal 2). During the interactions, T-cells produce IL-2 and enhance the expression of IL-2 receptors. Afterwards, IL-2-IL-2 receptor signaling occurs inside the T-cells (Signal 3). In order for the T-cells to be fully activated and perform immune function, all of the three signals are required. IL-2 receptors are composed of three types of protein; alpha, beta and gamma. Beta and gamma are always expressed whereas in the case of alpha, the amount of expression is adjusted according to the activation of T-cells.

Cyclosporin A, one of the most representative immunosuppressive, suppresses the generation of IL-2; it suppresses the function of a protein called calcineurin, which is concerned in Signals 1 and 2, and ultimately suppresses the function of NF-AT (a transcription factor) and the generation of IL-2. Since NF-AT is also concerned in the generation of IL-2 receptors, consequently, cyclosporin A suppresses the generation of IL-2 receptors.

3-1. Effect on expression of IL-2

T-cells were separated from the spleen of the C57BL/6 mouse for the experiment. 1 μg/m ConA was used to activate the T-cells. Prodigiosin and cyclosporin A were melted in DMSO and added to the culture medium in a manner to achieve a final concentration of 3, 10, 30 ng/ml. After 4 hours of cultivation, the cells were collected. After separating the RNA from the cells, the degree of expression of IL-2 mRNA was measured through RT-PCR (reverse transcription-polymerase chain reaction).

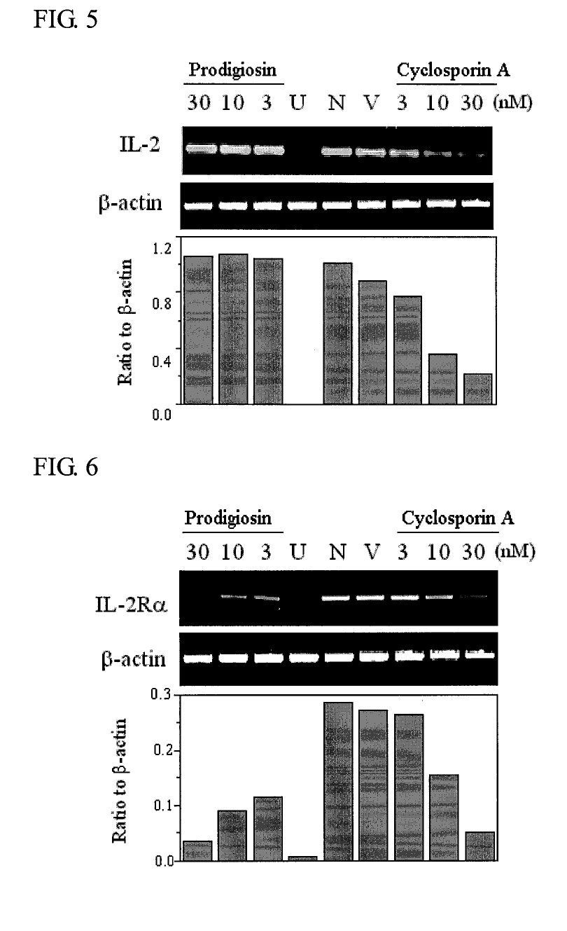

As seen in FIG. 5, the results of the experiment indicate that T-cells that were not activated could not express IL-2 mRNA (untreated group, U). However, when treated with ConA, the expression of IL-2 mRNA was significantly increased (Naive control, N). Meanwhile, 0.1% DMSO that was used as solvent did not influence the expression of IL-2 mRNA (vehicle control, V).

When 3, 10, 30 ng/ml prodigiosin was treated, it did not influence the expression of IL-2 mRNA, whereas cyclosporin A strongly suppressed the expression of IL-2 mRNA. RT-PCR was conducted on beta-actin, through which the same amount of RNA was used in the experiment. The graph shows the relative amount of beta-actin.

3-2. Effect on the expression of IL-2 receptor

T-cells were separated from the spleen of the C57BL/6 mouse for the experiment. 1 μg/ml ConA was used to activate the T-cells. Prodigiosin and cyclosporin A were melted in DMSO and added to the culture medium in a manner to achieve a final concentration of 3, 10, 30 ng/ml. After 4 hours of cultivation, the cells were collected. After separating the RNA from the cells, the degree of expression of IL-2 mRNA was measured through RT-PCR (Reverse Transcription-Polymerase Chain Reaction).

As seen in FIG. 6, the results of the experiment indicate that T-cells that were not activated could not express IL-2 mRNA (untreated group, U). However, when treated with ConA, the expression of IL-2 receptor mRNA was significantly increased (Naive control, N). Meanwhile 0.1% DMSO that was used as solvent did not influence the expression of IL-2 mRNA (vehicle control, V). When 3, 10, 30 ng/ml prodigiosin was treated, it efficiently suppressed the expression of IL-2 mRNA. Cyclosporin A also efficiently suppressed the expression of IL-2 mRNA. RT-PCR was conducted on beta-actin, through which the same amount of RNA was used in the experiment. The graph shows the relative amount of beta-actin.

The results of Experimental Examples 3-1 and 3-2 indicate that prodigiosin selectively suppresses the expression of IL-2 receptors. On the other hand, cyclosporin A successfully suppressed both IL-2 and IL-2 receptors. This proves that prodigiosin and cyclosporin A have different mode of actions.

Experimental Example 4. Relevance of the immunosuppressive effect of prodigiosin and cyclosporin to IL-2

4-1. Relation of the Immunosuppressive Effect of Prodigiosin to IL-2

T-cells were separated from the spleen of the C57BL/6 mouse for the experiment. 1 μg/ml ConA and 10 unit/ml IL-2 were used to generate the proliferation of T-cells. In the control group, 1 μg/ml ConA induced the proliferation of T-cells. Prodigiosin was melted in DMSO and added to the culture medium in a manner to achieve a final concentration of 3, 10, 30 ng/ml. After 60 hours of cultivation, [³H]-thymidine was added to the culture medium with a concentration of 1 μCi/well. After 12 hours, the cells were collected and the amount of radioactivity in the DNA was measured.

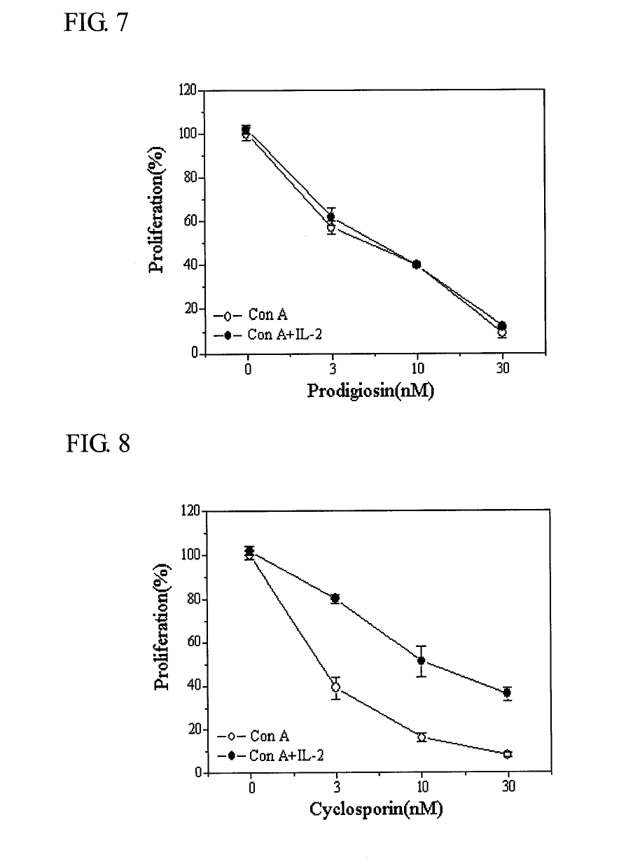

As seen in FIG. 7, the experimental results suggest that prodigiosin suppressed T-cells proliferated by ConA, while it did not suppress the excessive amount of IL-2 that was added from the outside.

4-2. Relation of the Immunosuppressive Effect of Cyclosporin a to IL-2

T-cells were separated from the spleen of the C57BL/6 mouse for the experiment. 1 μg/ml ConA and 10 unit/ml IL-2 were used to generate the proliferation of T-cells. In the control group, 1 μg/ml ConA induced the proliferation of T-cells. Cyclosporin A was melted in DMSO and added to the culture medium in a manner to achieve a final concentration of 3, 10, 30 ng/ml. After 60 hours of cultivation, [³H]-thymidine was added to the culture medium with a concentration of 1 μCi/well. After 12 hours, the cells were collected and the amount of radioactivity in the DNA was measured.

As seen in FIG. 8, the experimental results suggest that cyclosporin A suppressed T-cells proliferated by ConA, and was restored by the excessive amount of IL-2 that was added from the outside.

Thus, the results of Experimental Examples 4-1 and 4-2 show that the immunosuppressive effect of prodigiosin has no relevance to IL-2, whereas the immunosuppressive effect of cyclosporin A is restored by IL-2. This confirms the conclusion that prodigiosin and cyclosporin A have different mode of actions.

Experimental Example 5. Effect of Prodigiosin and Cyclosporin a on the Proliferation of IL-2 Dependent T-Cells

5-1. Effect of Prodigiosin on the Proliferation of IL-2 Dependent T-Cells

T-cells were separated from the spleen of the C57BL/6 mouse for the experiment. 1 μg/ml ConA was used to generate the proliferation of T-cells. (ConA-dependent T-cell proliferation).

In order to measure IL-2 dependent T-cell proliferation, T-cells separated from the spleen of the mouse were treated with 1 μg/ml ConA for 48 hours. Then, the cells were washed three times to get rid of all ConA for further experiment. The pre-treated cells have an excessive amount of IL-2 receptors so that they respond to IL-2 that are added from the outside, whereas the cells that are not pre-treated do not have IL-2 receptors to respond to the additional IL-2. 10 unit/ml IL-2 was added to the pre-treated cells to induce T-cell proliferation (IL-2-dependent T cell proliferation).

Prodigiosin was melted in DMSO and added to the culture medium in a manner to achieve a final concentration of 3, 10, 30 ng/ml. After 60 hours of cultivation, [³H]-thymidine was added to the culture medium with a concentration of 1 μCi/well. After 12 hours, the cells were collected and the amount of radioactivity in the DNA was measured.

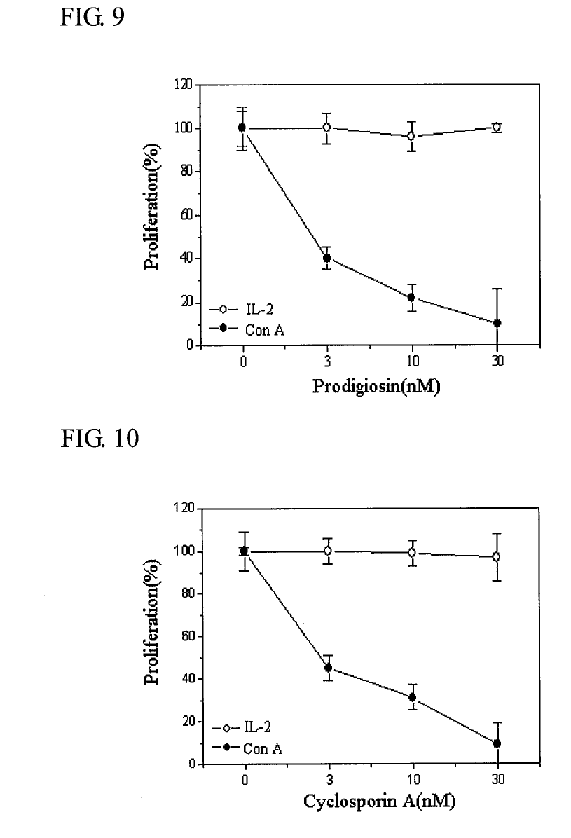

As seen in FIG. 9, the experimental results suggest that prodigiosin suppressed T-cells proliferated by ConA, while it did not suppress the T-cell proliferation by IL-2.

5-2. Effect of Cyclosporin a on the Proliferation of IL-2 Dependent T-Cells

T-cells were separated from the spleen of the C57BL/6 mouse for the experiment. 1 μg/ml ConA was used to generate the proliferation of T-cells. (ConA-dependent T-cell proliferation).

In order to measure IL-2 dependent T-cell proliferation, T-cells separated from the spleen of the mouse were treated with 1 μg/ml ConA for 48 hours. Then, the cells were washed three times to get rid of all the ConA for further experiment. The pre-treated cells have an excessive amount of IL-2 receptors so that they respond to IL-2 that are added from the outside, whereas the cells that are not pre-treated do not have IL-2 receptors to respond to the additional IL-2. 10 unit/ml IL-2 was added to the pre-treated cells to induce T-cell proliferation (IL-2-dependent T-cell proliferation).

Cyclosporin A was melted in DMSO and added to the culture medium in a manner to achieve a final concentration of 3, 10, 30 ng/ml. After 60 hours of cultivation, [³H]-thymidine was added to the culture medium with a concentration of 1 μCi/well. After 12 hours, the cells were collected and the amount of radioactivity in the DNA was measured.

As seen in FIG. 10, the experimental results suggest that cyclosporin A suppressed T-cells proliferated by ConA, while it did not suppress the T-cell proliferation by IL-2.

Thus, the results of Experimental Examples 5-1 and 5-2 suggest that prodigiosin and cyclosporin A do not influence IL-2-dependent T-cell proliferation, that is, the signal transduction after IL-2 is combined with its reception.

### INDUSTRIAL AVAILABILITY

Whereas the prodigiosin in this invention does not affect the expression of IL-2, it is immunosuppressive by selectively suppressing the proliferation of T-cells through the suppression of the expression of IL-2 receptors, which is needed in the activation of T-cells, while not affecting signal transduction after IL-2 receptors are combined with T-cells. Prodigiosin can be used either alone or in conjunction with cyclosporin A, a generally used immunosuppressive, for greater effect since the two substances have different mode of actions. These make prodigiosin effective for the prevention and treatment of acute graft-versus-host disease.

1. A composition for the prevention and treatment of acute graft-versus-host disease comprising prodigiosin isolated from Serratia marcescence B-1231 KCTC 0386BP, as an effective component.

2. The composition according to claim 1, the prodigiosin that has a different mode of action from that of cyclosporin A, which makes it available for use jointly for a more efficient prevention and treatment of acute graft-versus-host disease.
